(19) United States
(12) Patent Application Publication
(10) Pub. No.: US 2026/0108228 A1
(43) Pub. Date: Apr. 23, 2026

(54) METHOD FOR BREAST TUMOR BI-RADS CLASSIFICATION BASED ON ULTRASOUND AND COMPUTER DEVICE

(71) Applicant: Guangzhou Gexili Medical Technology Co., Ltd., Guangzhou City (CN)

(72) Inventors: Xiaochuan LI, Guangzhou City (CN); Zhenyu GUO, Guangzhou City (CN); Ningtao ZHANG, Guangzhou City (CN); Jingli LIU, Guangzhou City (CN)

(21) Appl. No.: 19/365,356

(22) Filed: Oct. 22, 2025

(30) Foreign Application Priority Data
Apr. 30, 2025 (CN) 202510564095.8
Oct. 22, 2024 (CN) 202411476529.0

(51) Int. Cl. A61B 8/08; G06F 18/25; G06T 7/00

(52) U.S. Cl. CPC A61B 8/0825; G06F 18/253; G06T 7/0012; G06T 2207/10132; G06T 2207/20081; G06T 2207/30068

(57) ABSTRACT

Provided are a method for breast tumor breast imaging reporting and data system (BI-RADS) classification based on ultrasound and a computer device. The method includes: processing an ultrasound radio frequency (RF) signal of a breast tumor to obtain a lesion location RF signal; processing the lesion location RF signal based on a feature extraction model to obtain a feature pixel matrix; and identifying and classifying the feature pixel matrix based on a preset strategy to obtain a class of the breast tumor. The method for breast tumor BI-RADS classification based on ultrasound provided herein can identify the ultrasound RF signal standardly using more efficient deep learning and intelligent classification and identification algorithms, retain more effective information, and finally obtain an identification result by imitating a method of scoring by a clinician based on signal features during identification and constraining each scoring result using a joint loss function.

### CROSS REFERENCE TO RELATED APPLICATION

This patent application claims the benefit and priority of Chinese Patent Application No. 202411476529.0, filed with the China National Intellectual Property Administration on October 22, 2024, and No. 202510564095.8, filed with the China National Intellectual Property Administration on April 30, 2025, the disclosure of which are incorporated by reference herein in their entirety as part of the present application.

### TECHNICAL FIELD

The present application relates to the field of data analysis, and in particular, to a method for breast tumor breast imaging reporting and data system (BI-RADS) classification based on ultrasound and a computer device.

### BACKGROUND

Breast cancer is the most common cancer in women. Approximately 1.2 million women are diagnosed with breast cancer each year with a mortality rate of 35% among confirmed patients. Breast tumor screening and tissue identification play a crucial role in reducing breast cancer mortality. The standard for breast tumor screening and tissue classification is Breast Imaging Reporting and Data System (BI-RADS). BI-RADS categories (hereinafter referred to as the “criteria”), originally proposed by the American College of Radiology in 1992, are used to predict potential risks based on detected pathologies in each case and serve as the primary criteria for assessing breast images and drawing conclusions. The criteria may typically include 7 categories (0 to 6), with class 4 being further subdivided into categories 4A, 4B, and 4C, collectively referred to as 9 categories herein. The challenge in identification based on the categories lies in similar features between adjacent categories, making it difficult to obtain objective and reproducible results even with comprehensive assessments from various perspectives. Consequently, in clinical practice, physicians rely on experience for standard assessments, which are often susceptible to subjectivity.

The most commonly used technologies in clinical tumor screening are ultrasound imaging and X-ray imaging. However, X-ray imaging performs poorly in screening and identifying breast lesions in women with dense breast tissue, with sensitivity dropping by 48–85% due to problems such as glandular obstruction. Ultrasound imaging, owing to its advantages in safety, patient tolerance, and dynamic assessment, has become the dominant means for breast cancer screening and identification. Nevertheless, clinical ultrasound imaging involves steps such as logarithmic compression, envelope addition, sampling, and denoising to ultrasound radio frequency (RF) signals, which inevitably lead to the loss of some effective information.

The foregoing description is intended to provide general background information and does not necessarily constitute the prior art.

### SUMMARY

In view of the above drawbacks of the prior art, an objective of the present application is to provide a method for breast tumor BI-RADS classification based on ultrasound and a computer device for solving the problems in the prior art, i.e., the loss of some effective information due to steps such as logarithmic compression, envelope addition, sampling, and denoising to ultrasound RF signals in clinical ultrasound imaging, and the susceptibility to subjectivity when physicians make standard assessments on breast tumors according to experience.

To achieve the above objective and other related objectives, the present application provides the following technical solutions.

In a first aspect, the present application provides a method for breast tumor BI-RADS classification based on ultrasound, including:

processing an ultrasound radio frequency (RF) signal of a breast tumor to obtain a lesion location RF signal;

processing the lesion location RF signal based on a feature extraction model to obtain a feature pixel matrix; and

identifying and classifying the feature pixel matrix based on a preset strategy to obtain a class of the breast tumor.

Preferably, the processing an ultrasound RF signal of a breast tumor to obtain a lesion location RF signal includes:

preprocessing the ultrasound RF signal to obtain an ultrasound image; and

processing the ultrasound image to obtain the lesion location RF signal.

Preferably, the preprocessing the ultrasound RF signal to obtain an ultrasound image includes:

acquiring information of a header file of the ultrasound RF signal;

correspondingly acquiring an ultrasound RF signal based on a byte offset of the header file; and

processing the ultrasound RF signal to obtain the ultrasound image.

Preferably, the processing the ultrasound image to obtain the lesion location RF signal includes:

acquiring annotation information of the ultrasound image, where the annotation information is provided to distinguish between a lesion and a background region in the ultrasound image;

binarizing the ultrasound image based on the annotation information to obtain a mask image that is binarized;

determining a lesion area in the ultrasound image based on the mask image;

determining a lesion boundary in the ultrasound image based on the lesion area;

expanding the lesion area in the ultrasound image based on the lesion boundary, where a location of the expanded lesion area in the ultrasound image is a target location; and

selecting an ultrasound RF signal corresponding to the target location as the lesion location RF signal.

Preferably, the processing the lesion location RF signal based on a feature extraction model to obtain a feature pixel matrix includes:

performing, by the feature extraction model, feature extraction based on the lesion location RF signal to obtain a feature map; and

performing fusion and stitching on the feature map to obtain the feature pixel matrix.

Preferably, the preset strategy includes:

performing stepwise classification on the feature pixel matrix by stepwise identification;

clustering classification results of the stepwise classification; and

processing the clustered classification results based on a benign class and a malignant class to obtain a final class.

Preferably, the performing stepwise classification on the feature pixel matrix by stepwise identification includes:

performing first-level identification on a classification result of the feature pixel matrix based on a first-level identification set, where the first-level identification set includes a first set BI-RADS class and a second set BI-RADS class;

performing second-level identification on the classification result of the feature pixel matrix based on a second-level identification set, where the second-level identification set includes a first sub-subset 1-1 BI-RADS class, a first sub-subset 1-2 BI-RADS class, a first sub-subset 1-3 BI-RADS class, a second sub-subset 2-1 BI-RADS class, a second sub-subset 2-2 BI-RADS class, and a second sub-subset 2-3 BI-RADS class;

performing third-level identification on the classification result of the feature pixel matrix based on a third-level identification set, where the third-level identification set includes a first sub-subset 1-1-1 BI-RADS class, a first sub-subset 1-1-2 BI-RADS class, a first sub-subset 1-1-3 BI-RADS class, a first sub-subset 1-1-4 BI-RADS class, a first sub-subset 1-2-1 BI-RADS class, a first sub-subset 1-2-2 BI-RADS class, a first sub-subset 1-2-3 BI-RADS class, a first sub-subset 1-2-4 BI-RADS class, and a first sub-subset 1-2-5 BI-RADS class; and

performing fourth-level identification on the classification result of the feature pixel matrix based on a fourth-level identification set, where the fourth-level identification set is obtained by re-clustering the first-level identification set, the second-level identification set, and the third-level identification set.

Preferably, the clustering classification results of the stepwise classification includes:

performing fifth-level identification on a result of the fourth-level identification based on a fifth-level identification set, where the fifth-level identification set is obtained by splitting the fourth-level identification set and includes a first set 5-1 BI-RADS class, a first set 5-2 BI-RADS class, a first set 5-3 BI-RADS class, and a first set 5-4 BI-RADS class.

Preferably, the processing the clustered classification results based on a benign class and a malignant class to obtain a final class includes:

performing sixth-level identification on the classification result of the feature pixel matrix based on a sixth-level identification set, where the sixth-level identification set is obtained by processing the fifth-level identification set based on the benign class and the malignant class and includes a benign class set and a malignant class set.

In a second aspect, the present application provides a computer device, including a memory, a processor, and a computer program stored on the memory and runnable on the processor, where the processor is configured to execute the computer program to implement the steps of the method for breast tumor BI-RADS classification based on ultrasound described above.

According to specific examples provided in the present application, the present application discloses the following technical effects:

The present application provides a method for breast tumor BI-RADS classification based on ultrasound and a computer device. The method can identify the ultrasound RF signal standardly using more efficient deep learning and intelligent classification and identification algorithms, retain more effective information, and finally obtain an identification result by imitating a method of scoring by a clinician based on signal features during identification and constraining each scoring result using a joint loss function.

### BRIEF DESCRIPTION OF THE DRAWINGS

To describe the technical solutions in the embodiments of the present application or in the related art more clearly, the following briefly describes the accompanying drawings required for describing the embodiments or the related art. Apparently, the accompanying drawings in the following description show some embodiments of the present application, and a person of ordinary skill in the art may still derive other accompanying drawings from these accompanying drawings without creative efforts.

FIG. 1 is a flowchart of a method for breast tumor BI-RADS classification based on ultrasound provided by an embodiment of the present application;

FIG. 2 is a flowchart of processing an ultrasound RF signal of a breast tumor to obtain a lesion location RF signal provided by an embodiment of the present application;

FIG. 3 is a flowchart of preprocessing an ultrasound RF signal to obtain an ultrasound image provided by an embodiment of the present application;

FIG. 4 is a flowchart of training a deep learning network provided by an embodiment of the present application;

FIGS. 5A-5H are schematic diagrams of an ultrasound image after expansion of a lesion area provided by an embodiment of the present application;

FIG. 6 is a flowchart of processing a lesion location RF signal provided by an embodiment of the present application; and

FIG. 7 is a flowchart of a stepwise identification method by an embodiment of the present application.

### DETAILED DESCRIPTION OF THE EMBODIMENTS

The technical solutions in the embodiments of the present application will be described clearly and completely below with reference to the accompanying drawings in the embodiments of the present application. Apparently, the described embodiments are merely some rather than all of the embodiments of the present application. All other embodiments derived from the embodiments in the present application by those of ordinary skill in the art without creative efforts should fall within the protection scope of the present application.

To make the above objective, features, and advantages of the present application more obvious and easier to understand, the present application will be further described in detail with reference to the accompanying drawings and specific implementations.

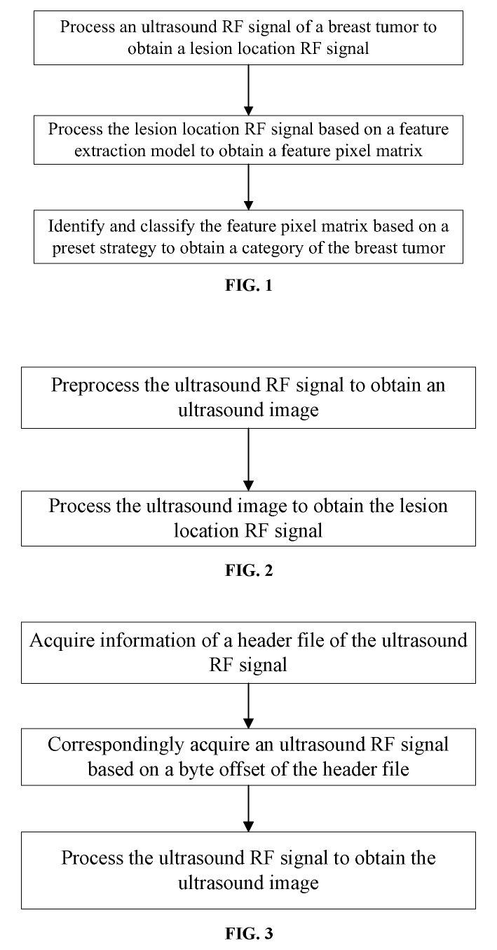

With reference to FIG. 1, the present application provides a method for breast tumor BI-RADS classification based on ultrasound, including the following steps.

An ultrasound RF signal of a breast tumor is processed to obtain a lesion location RF signal.

In an embodiment of the present application, to facilitate effective identification and classification in a screening and identification task for breast tumor tissue and improve the accuracy of identification and classification, effective feature extraction may be performed in advance on the lesion location RF signal in the ultrasound RF signal of the breast tumor. In this embodiment, A breast ultrasound RF signal is acquired by an ultrasound device scanning a patient’s breast. Therefore, the acquired breast ultrasound RF signal typically includes an ultrasound RF signal of a healthy location. It is more complicated that the ultrasound RF signal of the healthy location and the ultrasound RF signal of the breast tumor location are mixed in the acquired breast ultrasound RF signal. Hence, before classifying the patient’s breast tumor, the lesion location RF signal in the acquired breast ultrasound RF signal should be extracted for subsequent identification and classification.

The lesion location RF signal is processed based on a feature extraction model to obtain a feature pixel matrix.

In an embodiment of the present application, the lesion location RF signal is acquired and then input to the feature extraction model such that the feature extraction model selects and extracts the most effective features and attributes from the lesion location RF signal. The features extracted from the lesion location RF signal are further processed so as to identify and classify the breast tumor in subsequent steps.

The feature pixel matrix is identified and classified based on a preset strategy to obtain a class of the breast tumor.

In an embodiment of the present application, a stepwise identification strategy is adopted for identifying and classifying the acquired feature pixel matrix of the lesion location. That is, a plurality of highly similar categories with a low hazard level are classified into one class, while categories with a high hazard level are classified into another class. The classified categories are subdivided with respect to similarity. Finally, the classification of a plurality of categories (e.g., nine categories) is completed. Based on the divided categories, the acquired feature pixel matrix is identified stepwise, thereby completing classification.

With reference to FIG. 2, in an embodiment of the present application, the step of processing the ultrasound RF signal of the breast tumor to obtain the lesion location RF signal includes the following steps.

The ultrasound RF signal is preprocessed to obtain an ultrasound image.

In this embodiment, the ultrasound RF signal of the breast tumor is acquired by a clinical ultrasound device scanning the patient’s breast lesion. when extracting the breast ultrasound RF signal data, information in a header file needs to be determined, such as a number of single-frame RF data scanning lines, a number of sampling points, a center frequency of a probe, and a sampling rate. A starting point of the data can be localized by determining a byte offset of the header file such that the ultrasound RF signal data from the breast tumor is read accurately. After obtaining the RF signal, the RF signal is further subjected to processing such as logarithmic compression, envelope addition, and denoising, thereby obtaining the ultrasound image the breast ultrasound RF signal is mapped into.

With reference to FIG. 3, the step of preprocessing the ultrasound RF signal to obtain the ultrasound image includes the following steps.

Information of a header file of the ultrasound RF signal is acquired.

An ultrasound RF signal is correspondingly acquired based on a byte offset of the header file.

The ultrasound RF signal is processed to obtain the ultrasound image.

The ultrasound image is processed to obtain the lesion location RF signal.

In an embodiment of the present application, the acquired ultrasound image is input to a preset deep learning network such that the deep learning network extracts the ultrasound RF signal of the lesion location. The ultrasound RF signal of the lesion location includes a first plane and a second plane, with the lesion location being present in each plane.

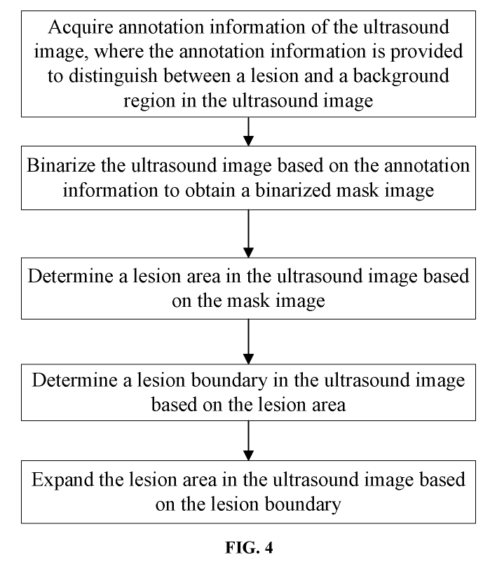

In this embodiment, when processing the ultrasound image by the deep learning network, the deep learning network needs to be trained in advance in a way as shown in FIG. 4.

Annotation information of the ultrasound image is acquired, where the annotation information is provided to distinguish between a lesion and a background region in the ultrasound image.

The ultrasound image is binarized based on the annotation information to obtain a binarized mask image.

A lesion area in the ultrasound image is determined based on the mask image.

A lesion boundary in the ultrasound image is determined based on the lesion area.

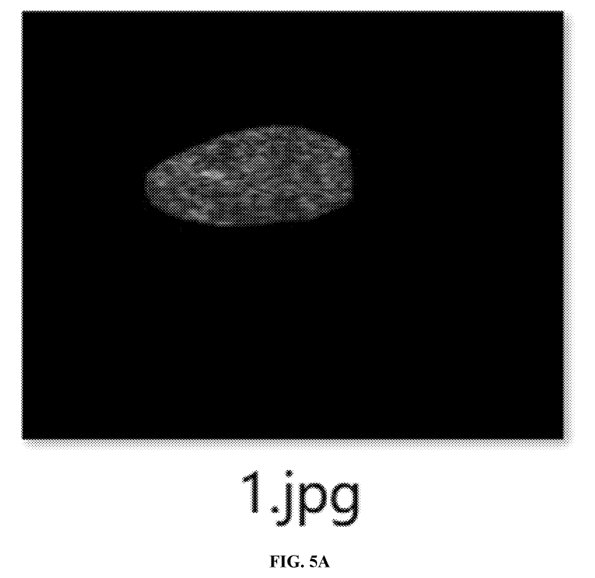

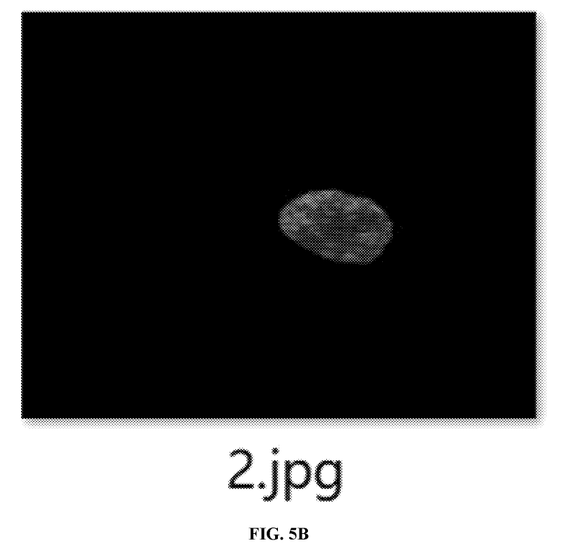

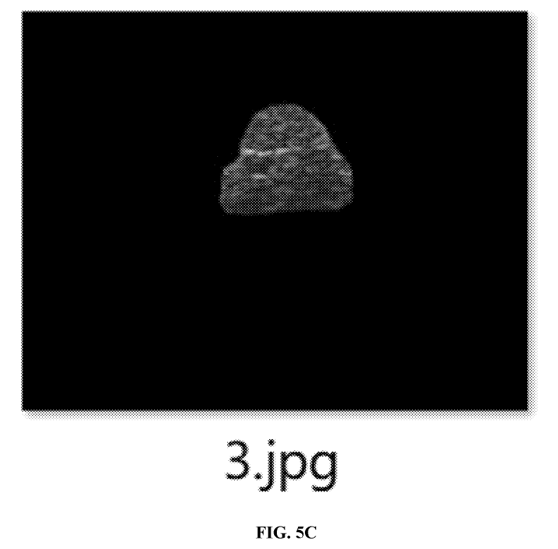

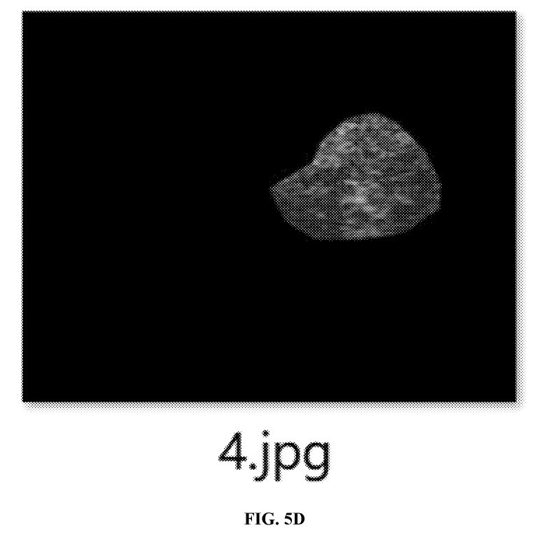

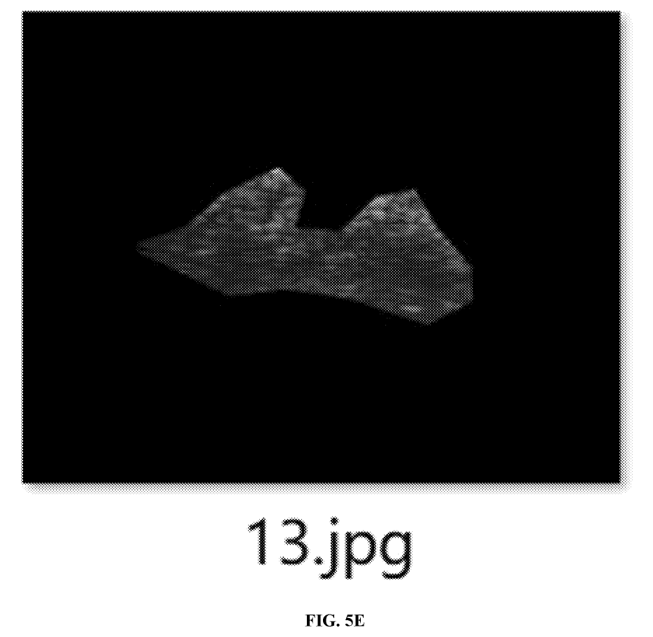

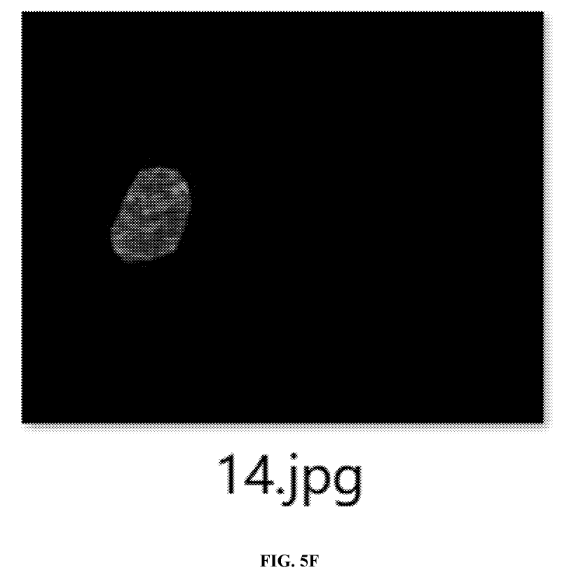

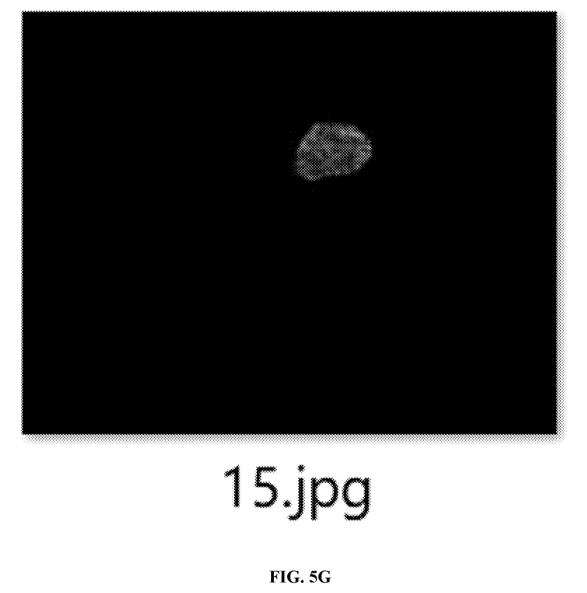

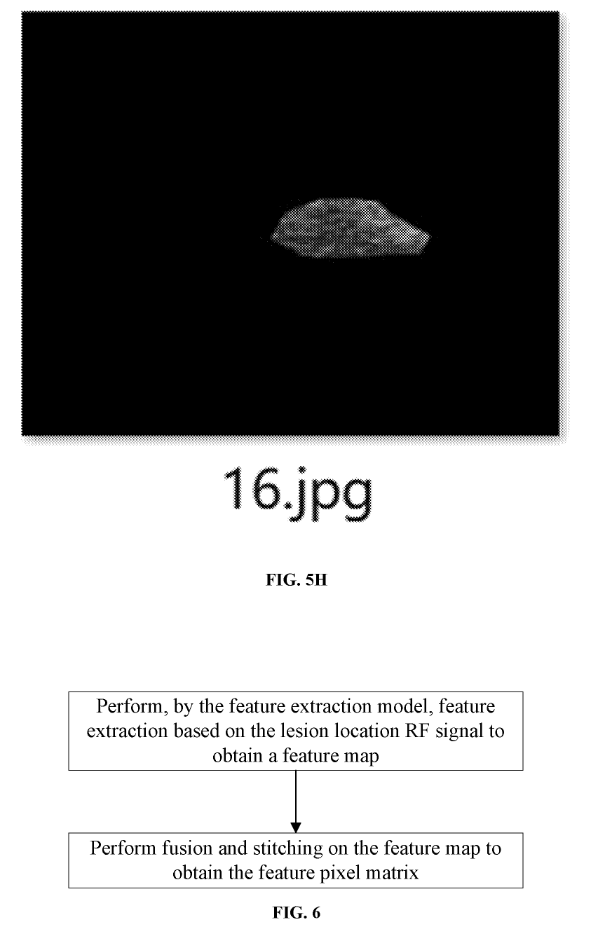

The lesion area in the ultrasound image is expanded based on the lesion boundary. The ultrasound image after expansion of the lesion area is as shown in FIGS. 5A-5H. A location of the expanded lesion area in the ultrasound image is a target location.

An ultrasound RF signal corresponding to the target location is selected as the lesion location RF signal.

The annotation information of the ultrasound image is acquired, and the ultrasound image is binarized based on the annotation information to obtain the binarized mask image. In this embodiment, the annotation information acquired from the ultrasound image includes lesion information and background information. During binarization, the ultrasound image is split based on the lesion information and the background region information to obtain the mask image. Further, the lesion area in the ultrasound image is determined based on the mask image and the lesion boundary is further determined such that the expanded lesion area is determined based on the lesion boundary. Still further, the target location of the lesion area in the ultrasound image, i.e., the lesion location, is determined based on the expanded ultrasound image. Further, the ultrasound RF signal of the target location is determined as the lesion location RF signal. A large quantity of historical ultrasound image information and the corresponding lesion location RF signal data are sent to the deep learning network for training. The trained deep learning network may process a newly input ultrasound image in the above way. Thus, the lesion location RF signal is obtained.

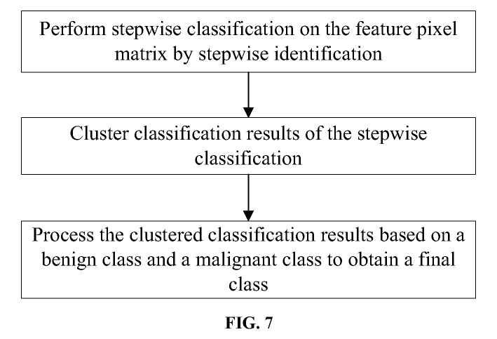

With reference to FIG. 6, the step of processing the lesion location RF signal based on the feature extraction model to obtain the feature pixel matrix includes the following steps.

Feature extraction is performed by the feature extraction model based on the lesion location RF signal to obtain a feature map.

Fusion and stitching are performed on the feature map to obtain the feature pixel matrix.

In an embodiment of the present application, after the lesion location RF signal is obtained by the above step, the lesion location RF signal further input to the feature extraction model. In this embodiment, the feature extraction model is a two-channel convolution model. Feature extraction is performed on the RF data of the RF signal in two planes with the same convolution weight to obtain feature maps that characterize plane characteristics and common lesion characteristics. Further, the feature maps respectively obtained through two channels are fused, i.e., stitched, forming a new feature pixel matrix of which the length remains unchanged and the width is doubled.

With reference to FIG. 7, the flow of the preset strategy is as follows.

Stepwise classification is performed on the feature pixel matrix by stepwise identification.

Classification results of the stepwise classification are clustered.

The clustered classification results are processed based on a benign class and a malignant class to obtain a final class.

In an embodiment of the present application, criteria for breast tumors are divided into 9 levels, namely class 0, class 1, class 2, class 3, class 4A, class 4B, class 4C, class 5, and class 6. Before classifying and identifying the acquired feature pixel matrix, the 9 categories need to be differently classified stepwise according to different levels. In this embodiment, during first-level identification on a classification result of the feature pixel matrix, the 9 categories are first divided into two classes, each corresponding to one set, namely a first set BI-RADS class and a second set BI-RADS class, where the first set BI-RADS class includes class 0, class 1, class 2, and class 3, and the second set BI-RADS class includes class 4A, class 4B, class 4C, class 5, and class 6. The first-level identification is performed on the feature pixel matrix based on the categories of the two set BI-RADS classes, with a binary cross entropy as a loss function and Sigmoid as an activation function.

During second-level identification on the classification result of the feature pixel matrix, the 9 categories are divided into 6 set BI-RADS classes, namely a first sub-subset 1-1 BI-RADS class, a first sub-subset 1-2 BI-RADS class, a first sub-subset 1-3 BI-RADS class, a second sub-subset 2-1 BI-RADS class, a second sub-subset 2-2 BI-RADS class, and a second sub-subset 2-3 BI-RADS class, where the first sub-subset 1-1 BI-RADS class includes class 0, the first sub-subset 1-2 BI-RADS class includes class 1, and the first sub-subset 1-3 BI-RADS class includes class 2 and class 3; the second sub-subset 2-1 BI-RADS class includes class 4A, class 4B, and class 4C, the second sub-subset 2-2 BI-RADS class includes class 5, and the second sub-subset 2-3 BI-RADS class includes class 6. The second-level identification is performed on the feature pixel matrix based on the categories of the six set BI-RADS classes, with a binary cross entropy as a loss function and Sigmoid as an activation function.

During third-level identification on the classification result of the feature pixel matrix, the 9 categories are divided into 9 set BI-RADS classes, namely a first sub-subset 1-1-1 BI-RADS class, a first sub-subset 1-1-2 BI-RADS class, a first sub-subset 1-1-3 BI-RADS class, a first sub-subset 1-1-4 BI-RADS class, a first sub-subset 1-2-1 BI-RADS class, a first sub-subset 1-2-2 BI-RADS class, a first sub-subset 1-2-3 BI-RADS class, a first sub-subset 1-2-4 BI-RADS class, and a first sub-subset 1-2-5 BI-RADS class, where the first sub-subset 1-1-1 BI-RADS class includes class 0, the first sub-subset 1-1-2 BI-RADS class includes class 1, the first sub-subset 1-1-3 BI-RADS class includes class 2, the first sub-subset 1-1-4 BI-RADS class includes class 3, the first sub-subset 1-2-1 BI-RADS class includes class 4A, the first sub-subset 1-2-2 BI-RADS class includes class 4B, the first sub-subset 1-2-3 BI-RADS class includes class 4C, the first sub-subset 1-2-4 BI-RADS class includes class 5, and the first sub-subset 1-2-5 BI-RADS class includes class 6. The third-level identification is performed on the feature pixel matrix based on the 9 set BI-RADS classes, with a binary cross entropy as a loss function and Sigmoid as an activation function.

During fourth-level identification on the classification result of the feature pixel matrix, the first-level identification set, the second-level identification set, and the third-level identification set are re-clustered. That is, the first set BI-RADS class, the second set BI-RADS class, the first sub-subset 1-1 BI-RADS class, the first sub-subset 1-2 BI-RADS class, the first sub-subset 1-3 BI-RADS class, the second sub-subset 2-1 BI-RADS class, the second sub-subset 2-2 BI-RADS class, the second sub-subset 2-3 BI-RADS class, the first sub-subset 1-1-1 BI-RADS class, the first sub-subset 1-1-2 BI-RADS class, the first sub-subset 1-1-3 BI-RADS class, the first sub-subset 1-1-4 BI-RADS class, the first sub-subset 1-2-1 BI-RADS class, the first sub-subset 1-2-2 BI-RADS class, the first sub-subset 1-2-3 BI-RADS class, the first sub-subset 1-2-4 BI-RADS class, and the first sub-subset 1-2-5 BI-RADS class are re-clustered into 9 categories, namely 9 categories in the classification criteria for breast tumors. The fourth-level identification is performed on the feature pixel matrix, with a binary cross entropy as a loss function and Sigmoid as an activation function.

Further, set classes corresponding to fifth-level identification are constructed based on the set BI-RADS classes corresponding to the fourth-level identification, namely a first set 5-1 BI-RADS class, a first set 5-2 BI-RADS class, a first set 5-3 BI-RADS class, and a first set 5-4 BI-RADS class, where the first set 5-1 BI-RADS class includes class 0 and class 1, the first set 5-2 BI-RADS class includes class 2 and class 3, the first set 5-3 BI-RADS class includes class 4A, class 4B, and class 4C, and the first set 5-4 BI-RADS class includes class 5 and class 6. The fifth-level identification is performed on the feature pixel matrix with the four sets, with a binary cross entropy as a loss function and Sigmoid as an activation function.

Further, set classes corresponding to sixth-level identification are formed by classifying the set classes corresponding to the fifth-level identification, namely a benign class and a malignant class. The feature pixel matrix is identified and classified based on the malignant class and the malignant class, with a binary cross entropy as a loss function and Sigmoid as an activation function.

In an embodiment of the present application, in order to further improve the identification accuracy, the above-mentioned non-interfering loss functions are assigned weights respectively, and combined, e.g., in a weighted summation manner, thereby obtaining an overall joint loss function. The joint loss function can achieve better overall performance through the performance of each u task and the coordination of a plurality of tasks. In this embodiment, the weight of each loss function is defined as follow:

(1) The loss function for the first-level two-class identification is set to 30%.

(2) The loss function for the two second-level three-class identifications is set to 15% and 15%.

(3) The loss function for the third-level four-class identification and five-class identification is set to 10% and 10%.

(4) The loss function for the fourth-level 9-class identification is set to 10%.

(5) The loss function for the fifth-level 4-class identification is set to 5%.

(6) The loss function for the sixth-level 2-class identification is set to 5%.

The identification results of the categories are integrated as the final identified class of the breast tumor for outputting.

In conclusion, the method for breast tumor BI-RADS classification based on ultrasound of the present application achieves high-accuracy breast tissue analysis from the ultrasound RF signal. This algorithm solves the problems of the traditional classification and analysis method, such as loss of effective information (more information is retained by using the RF signal), interference between categories (by stepwise identification, mutual interference between different class intervals is avoided, and the sensitivity of the network to different class intervals is enhanced by stepwise clustering after stepwise subdivision), unclear processing strategy (similar categories are identified stepwise before subdivision), difficult network convergence (the model is optimized by optimizing a plurality of loss functions together and using convergence criteria of different scales for categories of different class intervals), poor generalization performance (categories are stepwise clustered after being stepwise subdivided into subclasses, and finally regressed to the benign class and the malignant class. On the one hand, the repeatability and reversibility of identification are enhanced; and on the other hand, the previous classification results are fed back and checked using the subsequent architecture), and poor identification accuracy (this method performs well in classification based on BI-RADS).

In an exemplary embodiment, the present application further provides a computer device, including a memory, a processor, and a computer program stored on the memory and runnable on the processor, where the processor is configured to execute the computer program to implement the steps of the method for breast tumor BI-RADS classification based on ultrasound described above.

In an exemplary embodiment, provided is a computer-readable storage medium, storing a computer program which, when executed by a processor, implements the steps in the above embodiments of the method for breast tumor BI-RADS classification based on ultrasound.

In an exemplary embodiment, a computer device is provided. The computer device may be a server or a terminal. The computer device includes a processor, a memory, an input/output interface (I/O), and a communication interface. The processor, the memory, and the input/output interface are connected through a system bus, and the communication interface is connected to the system bus through the input/output interface. The processor of the computer device is configured to provide computing and control capabilities. The memory of the computer device includes a nonvolatile storage medium and an internal memory. The non-volatile storage medium stores an operating system, a computer program, and a database. The internal memory provides an environment for operation of the operating system and the computer program in the nonvolatile storage medium. The database of the computer device is configured to store data. The input/output interface of the computer device is configured to exchange information between the processor and an external device. The communication interface of the computer device is configured to communicate with an external terminal through a network. The computer program is configured to, when executed by the processor, implement a method for processing a breast ultrasound RF signal.

Those skilled in the art may understand that the structure described above is only a part of the structure related to the solutions of the present application and does not constitute a limitation on a computer device to which the solution of the present application is applied. Specifically, the computer device may include more or less components than those of the structure, or combine some components, or have different component arrangements.

In an exemplary embodiment, further provided is a computer device, including a memory and a processor, where the memory stores a computer program, and the computer program is executed by the processor to implement the steps in the above method embodiments.

In an exemplary embodiment, provided is a computer-readable storage medium, storing a computer program which, when executed by a processor, implements the steps in the above method embodiments.

In an exemplary embodiment, provided is a computer program product, including a computer program which, when executed by a processor, implements the steps in the above method embodiments.

It is to be noted that user information (including, but not limited to, user device information, user personal information, and the like) and data (including, but not limited to, data for analysis, data for storage, data for presentation, and the like) involved in the present application are information and data authorized by the user or fully authorized by each party, and relevant data shall be collected, used, and processed according to the related regulations.

Those of ordinary skill in the art may understand that all or some of the procedures in the methods of the above embodiments may be implemented by a computer program instructing related hardware. The computer program may be stored in a nonvolatile computer-readable storage medium. When the computer program is executed, the procedures in the embodiments of the above methods may be performed. Any reference to a memory, a database, or other media used in the embodiments of the present application may include a non-volatile and/or volatile memory. The nonvolatile memory may include a read-only memory (ROM), a magnetic tape, a floppy disk, a flash memory, an optical memory, a high-density embedded nonvolatile memory, a resistive random access memory (ReRAM), a magnetoresistive random access memory (MRAM), a ferroelectric random access memory (FRAM), a phase change memory (PCM), a graphene memory, etc. The volatile memory may include a random access memory (RAM) or an external cache memory. As an illustration rather than a limitation, the RAM may be in various forms, such as a static random access memory (SRAM) or a dynamic random access memory (DRAM).

The database in the embodiments of the present application may include at least one of a relational database and a non-relational database. The non-relational database may include a distributed database based on a blockchain, but is not limited thereto. The processor in the embodiments of the present application may be a general processor, a central processor, a graphics processor, a digital signal processor (DSP), a programmable logic device, and a data processing logic device based on quantum computing, but is not limited thereto.

The technical characteristics of the above embodiments can be employed in arbitrary combinations. To provide a concise description of these embodiments, all possible combinations of all the technical characteristics of the above embodiments may not be described; however, these combinations of the technical characteristics should be construed as falling within the scope defined by the specification as long as no contradiction occurs.

Several examples are used herein for illustration of the principles and implementations of the present application. The description of the foregoing examples is used to help illustrate the method of the present application and the core principles thereof. In addition, those of ordinary skill in the art can make various modifications in terms of specific implementations and scope of application in accordance with the teachings of the present application. In conclusion, the content of the present specification shall not be construed as a limitation to the present application.

What is claimed is:

1. A method for breast tumor breast imaging reporting and data system (BI-RADS) classification based on ultrasound, comprising:
processing an ultrasound radio frequency (RF) signal of a breast tumor to obtain a lesion location RF signal;
processing the lesion location RF signal based on a feature extraction model to obtain a feature pixel matrix; and
identifying and classifying the feature pixel matrix based on a preset strategy to obtain a class of the breast tumor.

2. The method for breast tumor BI-RADS classification based on ultrasound according to claim 1, wherein the processing an ultrasound RF signal of a breast tumor to obtain a lesion location RF signal comprises:
preprocessing the ultrasound RF signal to obtain an ultrasound image; and
processing the ultrasound image to obtain the lesion location RF signal.

3. The method for breast tumor BI-RADS classification based on ultrasound according to claim 2, wherein the preprocessing the ultrasound RF signal to obtain an ultrasound image comprises:
acquiring information of a header file of the ultrasound RF signal;
correspondingly acquiring an ultrasound RF signal based on a byte offset of the header file; and
processing the ultrasound RF signal to obtain the ultrasound image.

4. The method for breast tumor BI-RADS classification based on ultrasound according to claim 2, wherein the processing the ultrasound image to obtain the lesion location RF signal comprises:
acquiring annotation information of the ultrasound image, wherein the annotation information is provided to distinguish between a lesion and a background region in the ultrasound image;
binarizing the ultrasound image based on the annotation information to obtain a mask image that is binarized;
determining a lesion area in the ultrasound image based on the mask image;
determining a lesion boundary in the ultrasound image based on the lesion area;
expanding the lesion area in the ultrasound image based on the lesion boundary, wherein a location of the expanded lesion area in the ultrasound image is a target location; and
selecting an ultrasound RF signal corresponding to the target location as the lesion location RF signal.

5. The method for breast tumor BI-RADS classification based on ultrasound according to claim 1, wherein the processing the lesion location RF signal based on a feature extraction model to obtain a feature pixel matrix comprises:
performing, by the feature extraction model, feature extraction based on the lesion location RF signal to obtain a feature map; and
performing fusion and stitching on the feature map to obtain the feature pixel matrix.

6. The method for breast tumor BI-RADS classification based on ultrasound according to claim 1, wherein the preset strategy comprises:
performing stepwise classification on the feature pixel matrix by stepwise identification;
clustering classification results of the stepwise classification; and
processing the clustered classification results based on a benign class and a malignant class to obtain a final class.

7. The method for breast tumor BI-RADS classification based on ultrasound according to claim 6, wherein the performing stepwise classification on the feature pixel matrix by stepwise identification comprises:
performing first-level identification on a classification result of the feature pixel matrix based on a first-level identification set, wherein the first-level identification set comprises a first set BI-RADS class and a second set BI-RADS class;
performing second-level identification on the classification result of the feature pixel matrix based on a second-level identification set, wherein the second-level identification set comprises a first sub-subset 1-1 BI-RADS class, a first sub-subset 1-2 BI-RADS class, a first sub-subset 1-3 BI-RADS class, a second sub-subset 2-1 BI-RADS class, a second sub-subset 2-2 BI-RADS class, and a second sub-subset 2-3 BI-RADS class;
performing third-level identification on the classification result of the feature pixel matrix based on a third-level identification set, wherein the third-level identification set comprises a first sub-subset 1-1-1 BI-RADS class, a first sub-subset 1-1-2 BI-RADS class, a first sub-subset 1-1-3 BI-RADS class, a first sub-subset 1-1-4 BI-RADS class, a first sub-subset 1-2-1 BI-RADS class, a first sub-subset 1-2-2 BI-RADS class, a first sub-subset 1-2-3 BI-RADS class, a first sub-subset 1-2-4 BI-RADS class, and a first sub-subset 1-2-5 BI-RADS class; and
performing fourth-level identification on the classification result of the feature pixel matrix based on a fourth-level identification set, wherein the fourth-level identification set is obtained by re-clustering the first-level identification set, the second-level identification set, and the third-level identification set.

8. The method for breast tumor BI-RADS classification based on ultrasound according to claim 7, wherein the clustering classification results of the stepwise classification comprises:
performing fifth-level identification on the classification result of the feature pixel matrix based on a fifth-level identification set, wherein the fifth-level identification set is obtained by splitting the fourth-level identification set and comprises a first set 5-1 BI-RADS class, a first set 5-2 BI-RADS class, a first set 5-3 BI-RADS class, and a first set 5-4 BI-RADS class.

9. The method for breast tumor BI-RADS classification based on ultrasound according to claim 8, wherein the processing the clustered classification results based on a benign class and a malignant class to obtain a final class comprises:
performing sixth-level identification on the classification result of the feature pixel matrix based on a sixth-level identification set to obtain the class of the breast tumor, wherein the sixth-level identification set is obtained through classification based on the fifth-level identification set and comprises a benign class set and a malignant class set.

10. A computer device, comprising a memory, a processor, and a computer program stored on the memory and runnable on the processor, wherein the processor is configured to execute the computer program to implement steps of the method for breast tumor BI-RADS classification based on ultrasound according to claim 1.

11. The method for breast tumor BI-RADS classification based on ultrasound according to claim 6, wherein the processing an ultrasound RF signal of a breast tumor to obtain a lesion location RF signal comprises:
preprocessing the ultrasound RF signal to obtain an ultrasound image; and
processing the ultrasound image to obtain the lesion location RF signal.

12. The method for breast tumor BI-RADS classification based on ultrasound according to claim 11, wherein the preprocessing the ultrasound RF signal to obtain an ultrasound image comprises:
acquiring information of a header file of the ultrasound RF signal;
correspondingly acquiring an ultrasound RF signal based on a byte offset of the header file; and
processing the ultrasound RF signal to obtain the ultrasound image.

13. The method for breast tumor BI-RADS classification based on ultrasound according to claim 11, wherein the processing the ultrasound image to obtain the lesion location RF signal comprises:
acquiring annotation information of the ultrasound image, wherein the annotation information is provided to distinguish between a lesion and a background region in the ultrasound image;
binarizing the ultrasound image based on the annotation information to obtain a mask image that is binarized;
determining a lesion area in the ultrasound image based on the mask image;
determining a lesion boundary in the ultrasound image based on the lesion area;
expanding the lesion area in the ultrasound image based on the lesion boundary, wherein a location of the expanded lesion area in the ultrasound image is a target location; and
selecting an ultrasound RF signal corresponding to the target location as the lesion location RF signal.

14. The method for breast tumor BI-RADS classification based on ultrasound according to claim 6, wherein the processing the lesion location RF signal based on a feature extraction model to obtain a feature pixel matrix comprises:
performing, by the feature extraction model, feature extraction based on the lesion location RF signal to obtain a feature map; and
performing fusion and stitching on the feature map to obtain the feature pixel matrix.

15. The computer device according to claim 10, wherein the processing an ultrasound RF signal of a breast tumor to obtain a lesion location RF signal comprises:
preprocessing the ultrasound RF signal to obtain an ultrasound image; and
processing the ultrasound image to obtain the lesion location RF signal.

16. The computer device according to claim 15, wherein the preprocessing the ultrasound RF signal to obtain an ultrasound image comprises:
acquiring information of a header file of the ultrasound RF signal;
correspondingly acquiring an ultrasound RF signal based on a byte offset of the header file; and
processing the ultrasound RF signal to obtain the ultrasound image.

17. The computer device according to claim 15, wherein the processing the ultrasound image to obtain the lesion location RF signal comprises:
acquiring annotation information of the ultrasound image, wherein the annotation information is provided to distinguish between a lesion and a background region in the ultrasound image;
binarizing the ultrasound image based on the annotation information to obtain a mask image that is binarized;
determining a lesion area in the ultrasound image based on the mask image;
determining a lesion boundary in the ultrasound image based on the lesion area;
expanding the lesion area in the ultrasound image based on the lesion boundary, wherein a location of the expanded lesion area in the ultrasound image is a target location; and
selecting an ultrasound RF signal corresponding to the target location as the lesion location RF signal.

18. The computer device according to claim 10, wherein the processing the lesion location RF signal based on a feature extraction model to obtain a feature pixel matrix comprises:
performing, by the feature extraction model, feature extraction based on the lesion location RF signal to obtain a feature map; and
performing fusion and stitching on the feature map to obtain the feature pixel matrix.

19. The computer device according to claim 10, wherein the preset strategy comprises:
performing stepwise classification on the feature pixel matrix by stepwise identification;
clustering classification results of the stepwise classification; and
processing the clustered classification results based on a benign class and a malignant class to obtain a final class.

20. The computer device according to claim 19, wherein the performing stepwise classification on the feature pixel matrix by stepwise identification comprises:
performing first-level identification on a classification result of the feature pixel matrix based on a first-level identification set, wherein the first-level identification set comprises a first set BI-RADS class and a second set BI-RADS class;
performing second-level identification on the classification result of the feature pixel matrix based on a second-level identification set, wherein the second-level identification set comprises a first sub-subset 1-1 BI-RADS class, a first sub-subset 1-2 BI-RADS class, a first sub-subset 1-3 BI-RADS class, a second sub-subset 2-1 BI-RADS class, a second sub-subset 2-2 BI-RADS class, and a second sub-subset 2-3 BI-RADS class;
performing third-level identification on the classification result of the feature pixel matrix based on a third-level identification set, wherein the third-level identification set comprises a first sub-subset 1-1-1 BI-RADS class, a first sub-subset 1-1-2 BI-RADS class, a first sub-subset 1-1-3 BI-RADS class, a first sub-subset 1-1-4 BI-RADS class, a first sub-subset 1-2-1 BI-RADS class, a first sub-subset 1-2-2 BI-RADS class, a first sub-subset 1-2-3 BI-RADS class, a first sub-subset 1-2-4 BI-RADS class, and a first sub-subset 1-2-5 BI-RADS class; and
performing fourth-level identification on the classification result of the feature pixel matrix based on a fourth-level identification set, wherein the fourth-level identification set is obtained by re-clustering the first-level identification set, the second-level identification set, and the third-level identification set.
